(19) United States
(12) Patent Application Publication
(10) Pub. No.: US 2022/0204909 A1
(43) Pub. Date: Jun. 30, 2022

(54) MODULAR CONTINUOUS FLOW BIOREACTOR

(71) Applicant: HALTZAK BIOPHARMA LLC, Hillsborough, NC (US)

(72) Inventor: Oscar Alzate, Hillsborough, NC (US)

(21) Appl. No.: 17/564,820

(22) Filed: Dec. 29, 2021

Related U.S. Application Data
(60) Provisional application No. 63/132,105, filed on Dec. 30, 2020.

(51) Int. Cl. C12M 1/00; C12M 1/12; C12M 3/04; C12M 1/42

(52) U.S. Cl. CPC C12M 29/10; C12M 35/04; C12M 27/10; C12M 25/14

(57) ABSTRACT

Described herein is a modular continuous flow bioreactor for various cells. In one embodiment, the modular cell culture bioreactor apparatus may comprise a plurality of cell chambers disposed between an upper flow chamber and a lower flow chamber; a plurality of lower conduits fluidly connecting the lower flow chamber with one or more lower reservoirs and a plurality of upper conduits fluidly connecting the upper flow chamber with one or more upper reservoirs; one or more pumps fluidly connected through the plurality of conduits with the one or more reservoirs and with the upper and lower flow chambers; wherein each individual cell chamber comprises a lower permeable membrane in fluid communication with the lower flow chamber, a three-dimensional distribution of cells, and an upper permeable membrane in fluid communication with the upper flow chamber.

### CROSS REFERENCE TO RELATED APPLICATIONS

This application claims priority to U.S. Provisional Patent Application No. 63/132,105, filed on Dec. 30, 2020, which is incorporated by reference herein in its entirety.

### TECHNICAL FIELD

Described herein is a modular continuous flow bioreactor for various biological cell types. In one embodiment, the modular cell culture bioreactor apparatus may comprise a plurality of cell chambers disposed between an upper flow chamber and a lower flow chamber; a plurality of lower conduits fluidly connecting the lower flow chamber with one or more lower reservoirs and a plurality of upper conduits fluidly connecting the upper flow chamber with one or more upper reservoirs; one or more pumps fluidly connected through the plurality of conduits with the one or more reservoirs and with the upper and lower flow chambers; wherein each individual cell chamber comprises a lower permeable membrane in fluid communication with the lower flow chamber, a three-dimensional distribution of cells, and an upper permeable membrane in fluid communication with the upper flow chamber.

### BACKGROUND

The ability to culture in vitro viable three-dimensional cellular constructs that mimic natural tissue has proven challenging. This is because there are multiple dynamic biochemical interactions that take place between cells in vivo, many of which have yet to be fully understood. An important challenge of current biotechnology is producing cellular products for treatments in large quantities so that treatments can be more quickly produced. In particular, production of antibodies, vaccines, and metabolites has proven time- and resource-consuming. These are important limitations for a technology requiring significant advances to make it (1) more efficient, (2) cost-effective, (3) flexible, and (4) robust.

What is needed in the art is a system in which cells can be developed to form a three-dimensional construct contained inside millimeter-size bioreactors that can be modified, cleaned, replaced, and/or repaired without affecting the overall operativity of the system, thereby resulting in a flow cell-based system continuously generating cell products. Moreover, what is needed is a portable, scalable, modular apparatus to house these millimeter-sized cell cultures and produce cellular products on a large scale.

### SUMMARY

One embodiment described herein is a modular cell culture bioreactor apparatus. The modular cell culture bioreactor apparatus may comprise a plurality of cell chambers disposed between an upper flow chamber and a lower flow chamber; a plurality of lower conduits fluidly connecting the lower flow chamber with one or more lower reservoirs and a plurality of upper conduits fluidly connecting the upper flow chamber with one or more upper reservoirs; one or more pumps fluidly connected through the plurality of conduits with the one or more reservoirs and with the upper and lower flow chambers; wherein each individual cell chamber comprises a lower permeable membrane in fluid communication with the lower flow chamber, a three-dimensional distribution of cells, and an upper permeable membrane in fluid communication with the upper flow chamber. In some embodiments, the upper flow chamber, upper conduits, and at least one upper reservoir may comprise a culture medium. In some embodiments, the one or more upper reservoirs may comprise a culture medium, a cell feed, a cell supplement, a buffering agent, oxygen or carbon dioxide gases, or antibiotics. In some embodiments, the lower flow chamber, lower conduits, and at least one lower reservoir may comprise a buffered solution. In some embodiments, the three-dimensional distribution of cells may be embedded in a low-density gel. In some embodiments, the three-dimensional distribution of cells may be deposited with a 3D bioprinter. In some embodiments, the three-dimensional distribution of cells may be in solution or suspension. In some embodiments, the cells may comprise human, mammalian, insect, archaea, bacteria, cancers, genetically modified cells, or transformed or transfected cells. In some embodiments, the cell chamber may further comprise a layer of epithelial cells disposed upon the three-dimensional distribution of cells and under the upper permeable membrane. In some embodiments, the plurality of lower conduits and/or a plurality of upper conduits may comprise fluid or gas outlets, fluid or gas inlets, fluid waste ports, and sensors for temperature, pH, dissolved oxygen, or UV/Vis absorbance. In some embodiments, the plurality of lower conduits may be in fluid communication with a separation means.

Another embodiment described herein is a cell culture system comprising a plurality of the apparata as described herein that are fluidly connected to each other.

Another embodiment described herein is a method or means for cultivating cells and producing cellular products. The method may comprise introducing a plurality of cells into the bioreactor as described herein or the cell culture system as described herein and incubating the cells under conditions sufficient for growth. In an embodiment, the cellular products produced by the cells may be isolated.

Another embodiment described herein is a biological product produced by the method or means as described herein.

### DESCRIPTION OF THE DRAWINGS

FIG. 1A shows a top view idealized depiction of a bioreactor ensemble (100).

FIG. 1B shows a side view idealized depiction of a slice of the bioreactor ensemble that shows the bioreactor cell chambers (110).

FIG. 1C shows an idealized depiction of the cell chamber (110) that includes an upper permeable membrane (130) and a lower permeable membrane (150) with a three-dimensional distribution of cells (140) between the membranes.

FIG. 2A shows an idealized depiction of the ensemble-holding apparatus with the bioreactor ensemble (200). The straight arrows indicate the direction of fluid flow.

FIG. 2B shows an idealized depiction of the ensemble-holding apparatus without the bioreactor ensemble (270). The straight arrows indicate the direction of fluid flow.

FIG. 2C shows an idealized depiction of a three-dimensional rendition of the ensemble-holding apparatus without the bioreactor ensemble (270). The straight arrows indicate the direction of fluid flow.

FIG. 3 shows an idealized depiction of the modular cell culture bioreactor apparatus (300). The straight arrows indicate the direction of fluid flow. The semi-circular arrows indicate the direction of the pump flow. The upper fluid feeds the cells immobilized inside the cell chambers, while the lower fluid collects the cell products. The feeding fluid is sourced by feeding media of varied properties and conditions such as concentration of nutrients, buffers, or salts; whereas the lower fluid does not necessarily contain any of these ingredients and empties into a tangential flow column for sample removal. The cell products move from the feeding to the collecting fluid as a result of multiple gradients.

FIG. 4A shows an idealized depiction of a bioreactor unit (400, 450) with each bioreactor ensemble comprising a plurality of cell chambers placed horizontally (black-shaded areas 100) between the upper and lower flow chambers. For simplicity, three bioreactor ensembles are shown, however, a bioreactor unit can include fewer or greater than three bioreactor ensembles such as two to hundreds (or thousands) of bioreactor ensembles. Each ensemble is in contact with feeding fluid through the upper flow chamber and collection buffer through the lower flow chamber. The flow of the feeding fluid is indicated with grey lines and arrows and shown going from right to left. The feeding fluid enters on the right, feeds the cells inside the bioreactor ensemble, and goes out on the left. The collection fluid is shown with black lines and arrows and goes from left to right. The collection fluid enters on the left and exits on the right. However, in some embodiments, the feeding fluid may enter on the left and exit on the right while the collection fluid may enter on the right and exit on the left. The lower flow chamber can be connected with a tangential flow separation column and the sample is transported for downstream processing.

FIG. 4B shows an idealized depiction of the exterior of a bioreactor unit (450) with three bioreactor ensembles. The straight arrows indicate the direction of fluid flow.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present document, including definitions, will control. Preferred methods and materials are described below, although methods and materials similar or equivalent to those described herein can be used in practice or testing of the present invention.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or R) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a human. In another embodiment, the subject is a rat, mouse, or pig.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the term “contacting” refers to the placing of cells to be cultivated into a culture vessel with the medium and/or supplement in which the cells are to be cultivated. The term “contacting” encompasses inter alia mixing cells with medium and/or supplement, perfusing cells with medium and/or supplement, pipetting medium and/or supplement onto cells in a culture vessel, and submerging cells in culture medium and/or supplement.

As used herein, the term “cultivation” refers the maintenance of cells in an artificial environment under conditions favoring growth, differentiation, or continued viability, in an active or quiescent state, of the cells. Thus, “cultivation” may be used interchangeably with “cell culture,” “growing cells,” “maintaining cells,” or any of the synonyms described herein.

As used herein, the term “culture vessel” refers to a receptacle for holding cells. The vessel may be glass, plastic, metal, or other material that can provide an aseptic environment for culturing, holding, or storing cells. The culture vessel may be a plate with wells, such as a 6-well plate, 12-well plate, 24-well plate, or 96-well plate. The plate may be a non-tissue culture treated plate that can be used for a variety of cell culture applications. The plates may have a clear, untreated, hydrophobic polystyrene surface that is sterile. The plate may have a lid that is non-reversible and prevents cross-condensation among wells. The vessel can be a shaker flask, spinner flask, bioreactor, suspension bag, or other means for culturing cells. The term “container” is synonymous.

As used herein, the terms “cell culture” or “culture” refer to the maintenance of cells in an artificial, e.g., an in vitro environment. It is to be understood, however, that the term “cell culture” is a generic term and may be used to encompass the cultivation not only of cells such as human or animal cells, individual prokaryotic (e.g., bacterial) or eukaryotic (e.g., animal, plant, and fungal) cells, but also of tissues, human or animal tissues, organs, organ systems or whole organisms, for which the terms “tissue culture,” “organ culture,” “organ system culture” or “organotypic culture” may be used interchangeably with the term “cell culture.”

As used herein, the phrases “cell culture medium,” “culture medium,” “medium formulation,” “pluripotent stem cell composition,” or “medium” (plural “media” in each case) refer to a nutritive solution or “nutritive medium” that supports the cultivation and/or growth of cells; these phrases may be used interchangeably. A cell culture medium may be a basal medium (a general medium that requires additional ingredients to support cell growth) or a complete medium that has all or almost all components to support cell growth. Cell culture media may be serum-free, protein-free (one or both), may or may not require additional components like small molecules, growth factors, additives, feeds, supplements, for efficient and robust cell performance.

The term “combining” as used herein refers to the mixing or admixing of ingredients in a cell culture medium formulation. Combining can occur in liquid or powder form or with one or more powders and one or more liquids. In another example, two or more powdered components may be mixed and then agglomerated to produce a complex mixture such as media, media supplements, media subgroups, or buffers. Combining also includes mixing dry components with liquid components.

The phrases “concentrate feed supplement medium” or “concentrated feed supplement medium” are used interchangeably and refer to a medium that comprises at least one component that is at a concentration higher than that desired in the cell culture medium to be supplemented.

The term “extract” as used herein refers to a composition comprising or concentrated preparation of the subgroups of a substance, typically formed by treatment of the substance either mechanically (e.g., by pressure treatment) or chemically (e.g., by distillation, precipitation, enzymatic action or high salt treatment).

The term “effective amount” or “effective concentration” refers to an amount of an ingredient, which is available for use. One example is the amount of a vitamin in a culture medium, which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one skilled in the art and/or by experimental determination.

A “feed” or “supplement” as used herein refers to a composition when added to cells in standard culture may be beneficial for its maintenance, or expansion, or growth, or viability, or affects its cell performance, or increases culture longevity or maintaining cells in a pseudo-stationary phase wherein product expression continues, or results in a significant increase in final product titer. A feed or supplement may be used interchangeably in this disclosure and refers to tableted and liquid formats (including agglomerated formats) of media components comprising one or more amino acids, sugars, vitamins, buffers, sometimes, peptides, hydrolysates, fractions, growth factors, hormones, etc. required to rebalance or replenish or to modulate the growth or performance of a cell in culture, or a cell culture system. A feed or supplement may be distinguished from a cell culture medium in that it is added to a cell culture medium that can culture a cell. As would be understood by one of skill in the art, sometimes a feed/supplement may comprise mainly those amino acids, sugars, vitamins, buffers, etc. required to rebalance or replenish or modulate the growth or performance of a cell in culture, or a cell culture system. A feed or supplement may or may not be concentrated or may be partially concentrated for certain components only.

A cell culture medium is composed of a number of ingredients and these ingredients vary from one culture medium to another. A “1×formulation” is meant to refer to any aqueous solution that contains some or all ingredients found in a cell culture medium at working concentrations. The “1×formulation” can refer to, for example, the cell culture medium or to any subgroup of ingredients for that medium. The concentration of an ingredient in a 1×solution is about the same as the concentration of that ingredient found in a cell culture formulation used for maintaining or cultivating cells in vitro. A cell culture medium used for the in vitro cultivation of cells is a 1×formulation by definition. When a number of ingredients are present, each ingredient in a 1×formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic acid. A “1×formulation” of these amino acids contains about the same concentrations of these ingredients in solution. Thus, when referring to a “1×formulation,” it is intended that each ingredient in solution has the same or about the same concentration as that found in the cell culture medium being described. The concentrations and ingredients in a 1×formulation of cell culture medium is known to those of ordinary skill in the art. See Banes et al., Methods for Preparation of Media, Supplements and Substrate for Serum-Free Animal Cell Culture, Alan R. Liss, N.Y. (1984), which is incorporated by reference herein in its entirety. The osmolality and/or pH, however, may differ in a 1×formulation compared to the culture medium, particularly when fewer ingredients are contained in the 1×formulation. The 1×concentration of any component is not necessarily constant across various media formulations. 1×might therefore indicate different concentrations of a single component when referring to different media. However, when used generally, 1×will indicate a typical working concentration commonly found in the types of media being referenced. A 1×amount is the amount of an ingredient that will result in a 1×concentration for the relevant volume of medium.

A “10×formulation” as used herein refers to a solution wherein each ingredient in that solution is about 10 times more concentrated than the same ingredient in the cell culture medium. For example, a 10×formulation of RPMI-1640 culture medium may contain, among other ingredients, 2.0 g/L L-arginine, 0.5 g/L L-asparagine, and 0.2 g/L L-aspartic acid (compare 1×formulation, above). A “10×formulation” may contain a number of additional ingredients at a concentration about 10 times that found in the 1×culture medium. As will be readily apparent, “20×formulation,” “25×formulation,” “50×formulation” and “100×formulation” designate solutions that contain ingredients at about 20-, 25-, 50- or 100-fold concentrations, respectively, as compared to a working 1×cell culture medium. Again, the osmolality and pH of the media formulation and concentrated solution may vary. See U.S. Pat. No. 5,474,931, which is directed to culture media concentrate technology and is incorporated by reference herein for such teachings.

As used herein “physiologic pH” is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc. or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. Some supplements, though not preferred, may only be entirely soluble outside these ranges.

An “auto-pH” or “auto-pHing” medium, medium supplement, or buffer as described herein is a formulation which has been formulated such that, upon rehydration with a solvent, the resulting medium, medium supplement or buffer solution is at a desired pH and does not require adjustment of the pH with acid or base prior to use. For example, an auto-pH tableted culture medium that is formulated to be used at pH 7.4 will, upon rehydration with a solvent, be at pH 7.4 and therefore will be ready for immediate use without further adjustment of the pH.

The phrase “without significant loss of biological and biochemical activity” as used herein refers to a decrease of less than about 30%, preferably less than about 25%, more preferably less than about 20%, still more preferably less than about 15%, and most preferably less than about 10%, of the biological or biochemical activity of the nutritive media, media supplement, media subgroup, buffer, or sample of interest when compared to a freshly made nutritive media, media supplement, media subgroup, buffer, or sample of the same formulation.

As used herein a “solvent” is a liquid that dissolves or has dissolved another ingredient of the medium. Solvents may be used in preparing media, in preparing media tablets, in preparing tablets of subgroups, or supplements or other formulations, and in reconstituting a tablet or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five, six, or more components may be necessary in some cases to form a soluble mixture. Simple solvents such as mixtures of ethanol or methanol and water are preferred because of their ease of preparation and handling.

As used herein, “maintenance” refers generally to cells placed in a growth medium under conditions that facilitate cell growth, expansion, and/or division that may or may not result in a larger population of the cells.

As used herein “stem cells” refer to undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm, and ectoderm), as well as to give rise to tissues of multiple germ layers following transplantation and to contribute substantially to most, if not all, tissues following injection into blastocysts. Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multipotent stem cells; or (5) unipotent, meaning able to give rise to a single cell lineage (e.g., spermatogenic stem cells).

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

Various embodiments of the disclosure are described herein. It will be recognized that features specified in each embodiment may be combined with other specified features, including as indicated in the embodiments below, to provide further embodiments of the present disclosure.

Biologic drug manufacturing is traditionally conducted in large-scale, industrial bioreactors. However, there are challenges associated with large-scale, industrial bioreactors in addition to scaling up product production. The present disclosure addresses these issues by providing a disposable, modular bioreactor that offers potential advantages such as lower contamination risk, ease of handling, easy to assemble and transport, low carbon footprint, and time and cost savings. In addition, the complete system can be built using 3D printers, including casting the cell cultures within the cell chambers using 3D bioprinters.

Described herein is a system for producing and isolating cellular products in a continuous, modular fashion. The core of the device is a 3D distribution of cells (cell chambers), which are immobilized either by encapsulating immortalized cells in a low density biogel or containing the cells inside a cavity with permeable membranes. The cells inside the 3D distribution are continuously fed using a fluid (feeding fluid) that carries nutrients, growth and/or inhibition factors, pH regulators, gases, and other ingredients required to maintain a healthy cell culture according to the cell type. The lower section of the 3D distribution is in contact with another permeable membrane adjacent to a carrier fluid (collection fluid), which carries a buffer with ingredients such as phosphatase and protease inhibitors, or any other chemical required to maintain molecular stability and achieve proper separation. The composition of each fluid depends upon the cell type, the cell product, and other variables that can be determined by permanently monitoring the collection fluid. The cell products are collected out of the collection fluid using a tangential flow separation column. Further processing is utilized to isolate and purify cell products for biotechnological and pharmaceutical applications, such as drugs, metabolites, biologics, therapeutic proteins, etc. The system, including the apparata and cell deposition in the 3D distribution compartments, can be manufactured using 3D printers and 3D bioprinters, respectively. A cell chamber ensemble is the collection of arrays of cell chambers, constituting a cassette with hundreds of identical cell chambers. A scaled-up bioreactor will contain hundreds of these ensembles stacked on top of each other, separated by fluid chambers, thereby creating a bioreactor unit. Hundreds of units can be connected to create a factory. Each ensemble can be removed for cleaning or disposal, without affecting the operation of the factory. Inside the bioreactor unit, each ensemble can be removed and changed for a fresh ensemble, without affecting the operability of the unit. Such modularity offers the advantage of continuous operation and the 3D printing offers the advantage of cheap, high-quality permanent supply of units, and the recycling capacity offers a low carbon footprint. The ability to grow any cell type, creates a biologic factory adaptable to any cell product.

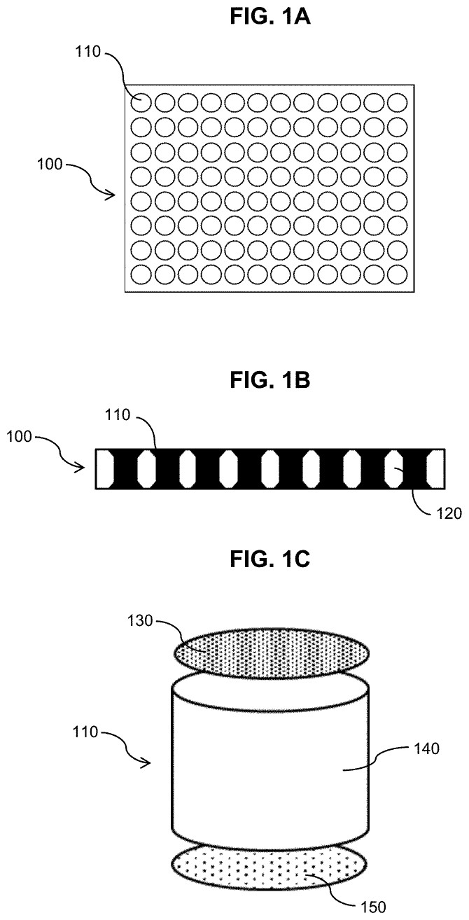

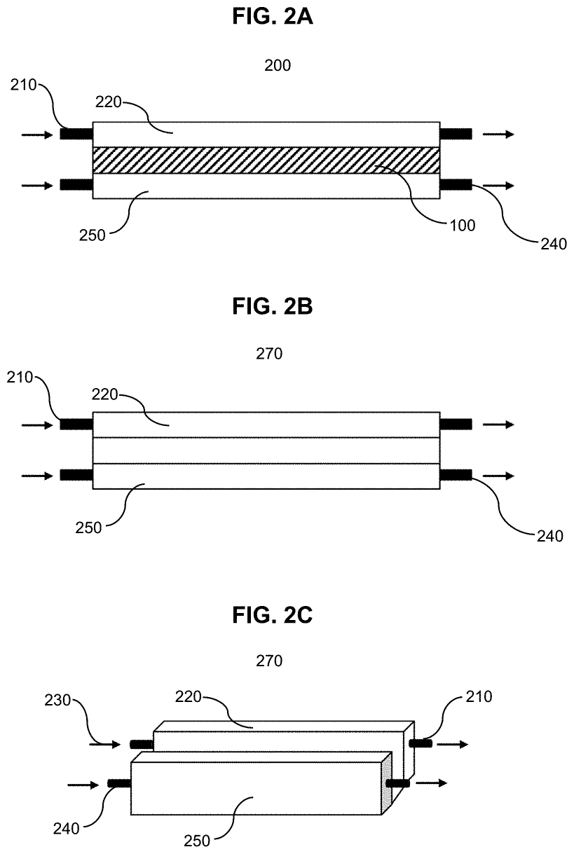

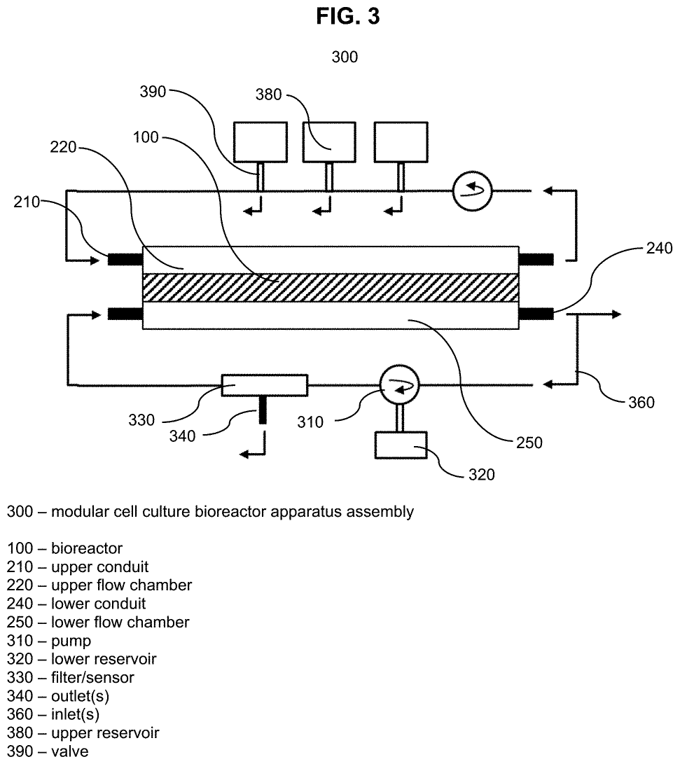

Described herein is a modular cell culture bioreactor 100 apparatus (300) that includes a plurality of cell chambers 110 disposed between an upper (e.g., feeding) flow chamber 220 and a lower (e.g., collection) flow chamber 250 (FIG. 1A-C; FIG. 2A-C; FIG. 3). The plurality of cell chambers 110 may be separated by spacers 120 (FIG. 1A-C). The foregoing description illustrates a horizontal configuration of the modular cell culture bioreactor 100 apparatus 300. The modular cell culture bioreactor can also be configured in a vertical orientation without any modifications to the embodiments described. The terms upper and lower are used throughout the description as applied to the horizontal configuration. In a vertical configuration, the terms “upper” or “lower” would be replaced with the terms “left” or “right,” as appropriate.

The cell chambers may contain immortalized cells that express a target product, such as a protein, peptide, metabolite, or any biological construct. The immortalized cells include, but are not limited to 3T3, HeLa, COS, 293/293T/HEK-293T, MDCK, CHO, S2, PC12, Neuro-2a/N2a, and SH-SY5Y cells. The cell chambers may contain non-immortalized cells that express a target product, such as a protein, peptide, metabolite, or any biological construct. The non-immortalized cells may include, but are not limited to, bacteria such as E. coli, Bacillus thuringiensis, Bacillus subtilis, hepatocytes, cardiomyocytes, or stem cells.

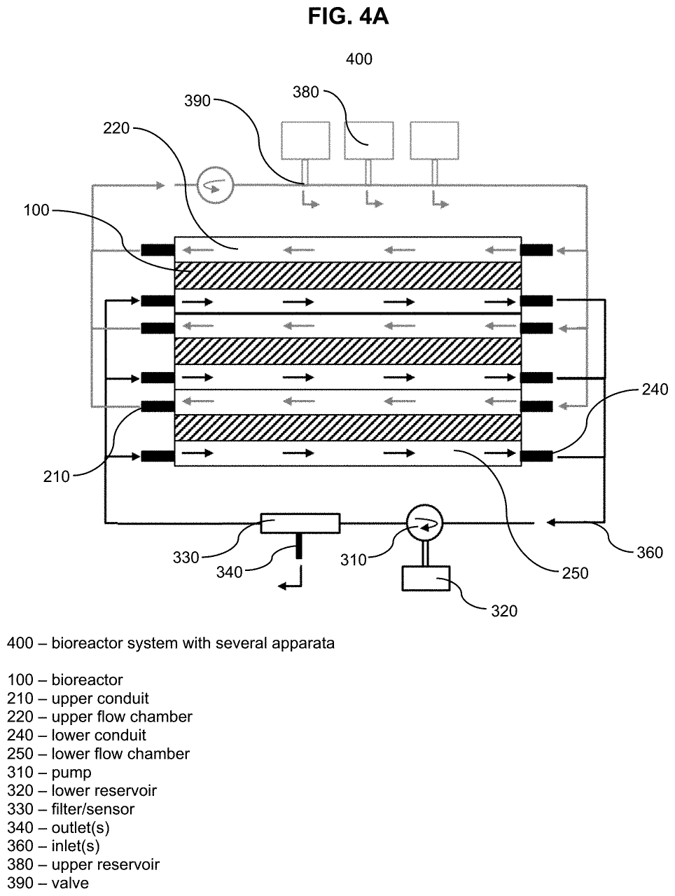

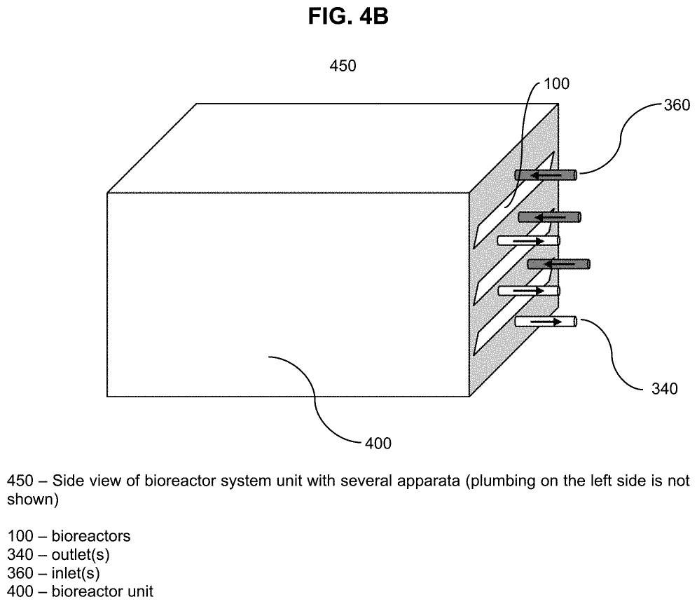

The modular cell culture bioreactor apparatus 300 further includes a plurality of lower conduits 240 fluidly connecting the collection flow chamber 250 with one or more lower reservoirs 380 and a plurality of upper conduits 210 fluidly connecting the feeding flow chamber 220 with one or more upper reservoirs 320 (FIG. 3). The reservoirs 320, 380 may be controlled by valves 390 that control the flow from the reservoirs 320, 380 into the conduits 210, 240. One or more pumps 310 are fluidly connected through the plurality of conduits 210, 240 with the one or more reservoirs 320, 380 and with the feeding 220 and collection flow chambers 250. Each individual cell chamber 110 includes a lower permeable membrane 150 in fluid communication with the collecting flow chamber 250; a three-dimensional distribution of cells 140; and an upper permeable membrane 130 in fluid communication with the feeding flow chamber 220. In some embodiments the upper permeable membrane 130 may be substituted for a layer of epithelial cells or a layer of epithelial cells are disposed immediately under the upper membrane 130. In the depiction of the modular cell culture bioreactor apparatus 300 in FIG. 3, the direction of the flow of the feeding solution (upper conduits and 220) and collection fluid (lower conduits and 250) are both shown traversing from left to right as an exemplary embodiment. The direction of the fluid flow of these upper and lower solutions can be independently controlled by their respective pumps 310 and may be in parallel (same direction, as shown in FIG. 3) or opposed (opposite directions, see FIG. 4). There may be advantages to either flow orientation (parrallel or opposed) that depends on the operation mode or stage; the flow direction may be changed throughout a production campaign to optimize production or achieve a desired result.

The bioreactor apparata 200 (FIG. 2A) can be stacked on top of each other, separated by their upper and lower fluid chambers, thereby creating a bioreactor unit 400, 450 (FIG. 4A-B). The bioreactor unit 400 includes a plurality of lower conduits 240 fluidly connecting the collection flow chamber 250 with one or more lower reservoirs 380 and a plurality of upper conduits 210 fluidly connecting the feeding flow chamber 220 with one or more upper reservoirs 320. The reservoirs 320, 380 may be controlled by valves 390 that control the flow from the reservoirs 320, 380 into the conduits 210, 240. One or more pumps 310 are fluidly connected through the plurality of conduits 210, 240 with the one or more reservoirs 320, 380 and with the feeding 220 and collection flow chambers 250. The flow of the feeding fluid is indicated with grey lines and arrows and shown going from right to left. The feeding fluid enters on the right, feeds the cells inside the bioreactor ensemble, and goes out on the left. The collection fluid is shown with black lines and arrows and goes from left to right. The collection fluid enters on the left and exits on the right. However, in some embodiments, the feeding fluid may enter on the left and exit on the right while the collection fluid may enter on the right and exit on the left. As described herein, the flow direction can be parallel or opposed and can be changed as needed to achieve a desired outcome. The lower flow chamber can be connected with a tangential flow separation column and the sample is transported for downstream processing.

The plurality of lower conduits and/or a plurality of lower conduits may include fluid or gas outlets 340, fluid or gas inlets 360, sensors 330 for temperature, pH, dissolved oxygen, UV/Vis absorbance, etc, and optional fluid waste ports. The plurality of lower conduits may be in fluid communication with a separation means (not shown). The separation means may be a tangential flow separation column, a size-exclusion column, a continuous centrifuge, a separation membrane, an affinity column, or any means known in the art capable of separating biological products from liquid media.

A bioreactor system may contain a plurality of the apparata as described herein that may be fluidly connected to each other (400, 450). The apparatus or cell culture system as described herein may be used for cultivating cells and producing cellular products.

One embodiment described herein is a modular cell culture bioreactor apparatus. The modular cell culture bioreactor apparatus may comprise a plurality of cell chambers disposed between an upper flow chamber and a lower flow chamber; a plurality of lower conduits fluidly connecting the lower flow chamber with one or more lower reservoirs and a plurality of upper conduits fluidly connecting the upper flow chamber with one or more upper reservoirs; one or more pumps fluidly connected through the plurality of conduits with the one or more reservoirs and with the upper and lower flow chambers; wherein each individual cell chamber comprises a lower permeable membrane in fluid communication with the lower flow chamber, a three-dimensional distribution of cells, and an upper permeable membrane in fluid communication with the upper flow chamber. In some embodiments, the upper flow chamber, upper conduits, and at least one upper reservoir may comprise a culture medium. In some embodiments, the one or more upper reservoirs may comprise a culture medium, a cell feed, a cell supplement, a buffering agent, oxygen or carbon dioxide gases, or antibiotics. In some embodiments, the lower flow chamber, lower conduits, and at least one lower reservoir may comprise a buffered solution. In some embodiments, the three-dimensional distribution of cells may be embedded in a low-density gel. In some embodiments, the three-dimensional distribution of cells may be deposited with a 3D bioprinter. In some embodiments, the three-dimensional distribution of cells may be in solution or suspension. In some embodiments, the cells may comprise human, mammalian, insect, archaea, bacteria, cancers, genetically modified cells, or transformed or transfected cells. In some embodiments, the cell chamber may further comprise a layer of epithelial cells disposed upon the three-dimensional distribution of cells and under the upper permeable membrane. In some embodiments, the plurality of lower conduits and/or a plurality of upper conduits may comprise fluid or gas outlets, fluid or gas inlets, fluid waste ports, and sensors for temperature, pH, dissolved oxygen, or UV/Vis absorbance. In some embodiments, the plurality of lower conduits may be in fluid communication with a separation means.

Another embodiment described herein is a cell culture system comprising a plurality of the apparata as described herein that are fluidly connected to each other.

Cells that are particularly amenable to cultivation as described herein include, but are not limited to, bacterial cells, fish cells, yeast cells, plant cells, and animal cells. Such bacterial cells, yeast cells, plant cells, or animal cells are available commercially from known culture depositories, e.g., the American Type Culture Collection (Manassas, Va.) and others that will be familiar to one of ordinary skill in the art. Preferred animal cells for cultivation by these methods include, but are not limited to, insect cells (most preferably Drosophila cells, Spodoptera cells and Trichoplusia cells), nematode cells (most preferably C. elegans cells) and mammalian cells (most preferably CHO cells, COS cells, VERO cells, BHK cells, AE-1 cells, SP2/0 cells, L5.1 cells, hybridoma cells and human cells, such as 293 cells, PER-C6 cells, or HeLa cells), any of which may be a somatic cell, a germ cell, a normal cell, a diseased cell, a transformed cell, a mutant cell, a stem cell, a precursor cell or an embryonic cell, embryonic stem cells (ES cells), cells used for virus or vector production (i.e., 293, PerC 6), cells derived from primary human sites used for cell or gene therapy, i.e., lymphocytes, hematopoietic cells, other white blood cells (WBC), macrophage, neutrophils, dendritic cells, and any of which may be an anchorage-dependent or anchorage-independent (i.e., “suspension”) cell. Another aspect is the manipulation or cultivation of cells and/or tissues for tissue or organ transplantation or engineering, i.e., hepatocyte, pancreatic islets, osteoblasts, osteoclasts/chondrocytes, dermal or muscle or other connective tissue, epithelial cells, tissues like keratinocytes, cells of neural origin, cornea, skin, organs, and cells used as vaccines, i.e., blood cells, hematopoietic cells other stem cells or progenitor cells, and inactivated or modified tumor cells of various histotypes.

The upper (feeding) flow chamber, upper conduits, and at least one upper reservoir may contain a culture medium. One or more of the upper reservoirs may contain a culture medium, a cell feed, a cell supplement, a buffering agent, oxygen or carbon dioxide gases, or antibiotics. The gases may also include inert gases such as nitrogen or argon. The lower (collection) flow chamber, lower conduits, and at least one lower reservoir may contain a buffered solution. The upper flow chamber feeds the cells inside the cell chamber. The lower flow chamber collects the cell products and delivers them to a tangential flow separation column for product separation and downstream processing. Downstream processing may include further separation by means known in the art, including filtration, precipitation, centrifugation, chromatography, or immunoprecipitation.

Examples of animal cell culture media that may be used as described herein include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, F10 Nutrient Mixture, F12 Nutrient Mixture, and cell-specific serum-free media (SFM) such as those designed to support the culture of keratinocytes, endothelial cells, hepatocytes, melanocytes, CHO cells, 293 cells, PerC6, hybridomas, hematopoetic cells, embryonic cells, neural cells etc. Specific chemically defined media products include CD CHO Medium (Gibco®), CD OptiCHO Medium (Gibco®), EX-CELLO Advanced™ CHO Medium (Millipore Sigma-Aldrich), HyClone™ ActiPro™ (GE Healthcare Life Sciences). Specific feed supplements include CHO CD EfficientFeed™ A (or B) AGT™ Nutritional Supplement (Gibco®), CD EfficientFeed™ C AGT™ Nutrient Supplement (Gibco®), EfficientFeed™ A+AGT™ Supplement (Gibco®), EfficientFeed™ B+AGT™ Supplement (Gibco®), Resurge™ CD1 Supplement (Gibco®), HyClone™ Cell Boost Supplements (various versions) (GE Healthcare Life Sciences), EX-CELLO Advanced™ CHO Feed 1 (Millipore Sigma-Aldrich), et al. Other media, media supplements, and media subgroups suitable for preparation are available commercially. Formulations for these media, media supplements and media subgroups, as well as many other commonly used animal cell culture media, media supplements and media subgroups are well-known in the art and are described in the literature and available from commercial suppliers, e.g., Thermo Fisher Scientific Inc., Life Technologies, Gibco, Invitrogen, et al.

Examples of plant cell culture media that may be used as described herein include, but are not limited to, Anderson's Plant Culture Media, CLC Basal Media, Gamborg's Media, Guillard's Marine Plant Culture Media, Provasoli's Marine Media, Kao and Michayluk's Media, Murashige and Skoog Media, McCown's Woody Plant Media, Knudson Orchid Media, Lindemann Orchid Media, or Vacin and Went Media. Formulations for these media, which are commercially available, as well as for many other commonly used plant cell culture media, are known in the art and available from commercial manufacturers.

Examples of bacterial cell culture media that may be used as described herein include, but are not limited to, Trypticase Soy Media, Brain Heart Infusion Media, Yeast Extract Media, Peptone-Yeast Extract Media, Beef Infusion Media, Thioglycollate Media, Indole-Nitrate Media, MR-VP Media, Simmons' Citrate Media, CTA Media, Bile Esculin Media, Bordet-Gengou Media, Charcoal Yeast Extract (CYE) Media, Mannitol-salt Media, MacConkey's Media, Eosin-methylene blue (EMB) media, Thayer-Martin Media, Salmonella-Shigella Media, and Urease Media. Formulations for these media, which are commercially available, as well as for many other commonly used bacterial cell culture media, are well-known in the art and may be found for example in the DIFCO™ & BBL™ Manual, 2^{nd} ed. (Becton, Dickinson and Company, 2009) and in the Manual of Clinical Microbiology (American Society for Microbiology, Washington, D.C.).

Examples of fungal cell culture media, particularly yeast cell culture media, that may be prepared as described herein include, but are not limited to, Sabouraud Media and Yeast Morphology Media (YMA). Formulations for these media are commercially available and are known in the art.

Examples of buffering agents that may be used herein include, but are not limited to, acetic acid, acetylsalicylic acid, adipic acid, alginic acid, ascorbic acid, aspartic acid, benzoic acid, benzenesulfonic acid, bisulfic acid, boric acid, butanoic acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, gly-glycine, gluco heptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthilic acid, nicotinic acid, nitrous acid, oxalic acid, pelargonic acid, phosphoric acid, propionic acid, pyruvic acid, saccharin, salicylic acid, sorbic acid, succinic acid, sulfuric acid, tartaric acid, thiocyanic acid, thioglycolic acid, thiosulfuric acid, tosylic acid, undecylenic acid, MES, bis-tris methane, ADA, ACES, bis-tris propane, PIPES, PBS, MOPSO, cholamine chloride, MOPS, BES, TES, HEPES, DIPSO, MOBS, acetamido glycine, TAPSO, TEA, POPSO, HEPPSO, EPS, HEPPS, Tricine, Tris(hydroxymethyl)aminomethane (tromethamine), glycinamide, glycylglycine, HEPBS, Bicine, TAPS, AMPB, CHES, AMP, AMPSO, CAPSO, CAPS, CABS, combinations thereof, or salts thereof. In one aspect, the buffer comprises one or more of phosphate, sulfate, carbonate, formate, acetate, propionate, butanoate, lactate, glycine, maleate, pyruvate, citrate, aconitate, isocitrate, a-ketoglutarate, succinate, fumarate, malate, oxaloacetate, aspartate, glutamate, tris(hydroxymethyl)aminomethane (tromethamine), combinations thereof, or salts thereof.

Examples of antibiotics include, but are not limited to, Geneticin™ carbenicillin, cefotaxime, anti-PPLO, Fungizone™, hygromycin, kanamycin, neomycin, nystatin, penicillin, or streptomycin, and the like, which are known in the art.

The three-dimensional distribution of cells may be embedded in a low-density gel or solution. A bioprinter may be used to deposit the cells. The low-density gel may have structural features that provide a matrix having a large surface area for cell adherence and yet porous enough to facilitate nutrient transport to the cells grown in the matrix. The low-density gel may comprise a biopolymer. Biopolymers suitable for use with the apparatus as described herein include any polymer that is gellable in situ, i.e. one that does not require chemicals or conditions (e.g. temperature, pH) that are not cytocompatible. This includes both stable and biodegradable biopolymers. Polymers that can be used in the apparatus as described herein include, but are not limited to, PEG hydrogels, alginate, agarose, collagen, hyaluronic acid (HA), peptide-based self-assembling gels, thermo-responsive poly(NIPAAm). Although some of the polymers listed are not innately light sensitive (e.g. collagen, HA), they may be made light sensitive by the addition of acrylate or other photosensitive groups. A number of biopolymers are known to those skilled in the art (Bryant and Anseth, 2001; Mann et al., 2001; and Peppas et al., 2000). Any cytocompatible polymer with the appropriate polymerization properties can be used. The selection of appropriate polymers is well within the ability of those skilled in the art. The biopolymers or solutions may additionally contain any of a number of growth factors, adhesion molecules, degradation sites or bioactive agents to enhance cell viability or for any of a number of other reasons. The biopolymers or the solutions may additionally contain adhesion inhibitors so as to keep the cells in solution. The biopolymers or the solutions may contain cell cycle inhibitors to prevent cell growth and/or differentiation. Such molecules are known in the art.

The three-dimensional distribution of cells may be deposited in layers. The three-dimensional distribution of cells may be deposited in about 2 layers, about 3 layers, about 4 layers, about 5 layers, about 6 layers, about 7 layers, about 8 layers, about 9 layers, or about 10 layers or up to about 100 layers on the lower permeable membrane. The cells may be deposited using a three-dimensional bioprinter. The cells may comprise, but are not limited to, any cell types including human, mammalian, insect, bacteria, archaea, plants, yeasts, fungi, cancer cells etc. The cell may be immortalized or naturally or artificially genetically modified. The cells may be transformed or transfected cells either permanently or transiently.

One exemplary cell type is human hepatocytes. Human hepatocytes are capable of producing cellular products such as those produced by a human liver. The cellular products include, but are not limited to, glucose, bile acids, cholesterol (e.g., LDL-C, VLDL), phospholipids, lipoproteins, ceruloplasmin, transferrin, complement factors, glycoproteins, and clotting factors (e.g., serum albumin, fibrinogen, prothrombin).

Bacteria can produce toxins (e.g., exotoxins such as botulinum toxin, Corynebacterium diphtheriae toxin, and tetanospasmin) and metabolites (e.g., primary, or secondary metabolites). The transformed or transfected cells can be engineered cells such as cells modified by DNA editing tools (e.g., rAAV, ZFN, CRISPR, and Transposon) that can model diseases or act as reporter cells with the use of fluorescent technology. Transformed or transfected cells can produce biologics such as proteins, nucleic acids, lipids, antibodies, and metabolites. Examples of proteins are inebilizumab, necitumumab, seribantumab, bevacizumab, cetuximab, otlertuzumab, ApoE, LDL, VLDL. Examples of nucleic acids are fomivirsen, mipomersen. Examples of metabolites are antibiotics (e.g., erythromycin A), antifungal agents (e.g., nystatin), anticancer agents (e.g., actinomycin), immunosuppressive agents (e.g., rapamycin), anti-inflammatory agents (e.g., salinamides A and B), biofilm inhibitory agents (e.g., cahuitamycins A-C), antiparasitic (e.g., avermectin).

The cell chamber may further include a layer of epithelial cells disposed upon the three-dimensional distribution of cells and under the upper permeable membrane. In some embodiments, the layer of epithelial cells may replace the upper permeable membrane. The epithelial cells may be deposited on the three-dimensional distribution of cells in a polarized configuration. The apical side of the epithelial cells may be in contact with the fluid from the upper flow chamber and the basal side may be in contact with the three-dimensional distribution of cells.

Also described herein are methods or means for cultivating cells and producing cellular products. The method includes introducing a plurality of cells into the bioreactor as described herein or the cell culture system as described herein and incubating the cells under conditions sufficient for growth. The conditions sufficient for growth are dependent on the cell type and the cellular products to be produced. Conditions that are sufficient for growth are known in the art. One of skill in the art can determine the conditions that are sufficient or optimal for growth and production of cellular products through routine experimentation. The cellular products produced by the cells may be isolated. Isolation of cellular products is known in the art and can be done by methods such as centrifugation, antibody selection, filtration, etc.

Further described herein are biological products produced by the method or means of as described herein.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

Various embodiments and aspects of the inventions described herein are summarized by the following clauses:

Clause 1. A modular cell culture bioreactor apparatus comprising:

a plurality of cell chambers disposed between an upper flow chamber and a lower flow chamber;

a plurality of lower conduits fluidly connecting the lower flow chamber with one or more lower reservoirs and a plurality of upper conduits fluidly connecting the upper flow chamber with one or more upper reservoirs;

one or more pumps fluidly connected through the plurality of conduits with the one or more reservoirs and with the upper and lower flow chambers;

wherein each individual cell chamber comprises a lower permeable membrane in fluid communication with the lower flow chamber; a three-dimensional distribution of cells; and an upper permeable membrane in fluid communication with the upper flow chamber.

Clause 2. The apparatus of clause 1, wherein the upper flow chamber, upper conduits, and at least one upper reservoir comprises a culture medium.
Clause 3. The apparatus of clause 1 or 2, wherein the one or more upper reservoirs comprise a culture medium, a cell feed, a cell supplement, a buffering agent, oxygen or carbon dioxide gases, or antibiotics.
Clause 4. The apparatus of any one of clauses 1-3, wherein the lower flow chamber, lower conduits, and at least one lower reservoir comprises a buffered solution.
Clause 5. The apparatus of any one of clauses 1-4, wherein the three-dimensional distribution of cells is embedded in a low-density gel.
Clause 6. The apparatus of any one of clauses 1-5, wherein the three-dimensional distribution of cells is deposited with a 3D bioprinter.
Clause 7. The apparatus of any one of clauses 1-4, wherein the three-dimensional distribution of cells is in solution or suspension.
Clause 8. The apparatus of any one of clauses 1-7, wherein the cells comprise human, mammalian, insect, archaea, bacteria, cancers, genetically modified cells, or transformed or transfected cells.
Clause 9. The apparatus of any one of clauses 1-8, wherein the cell chamber further comprises a layer of epithelial cells disposed upon the three-dimensional distribution of cells and under the upper permeable membrane.
Clause 10. The apparatus of any one of clauses 1-9, wherein the plurality of lower conduits and/or a plurality of upper conduits comprise fluid or gas outlets, fluid or gas inlets, fluid waste ports, and sensors for temperature, pH, dissolved oxygen, or UV/Vis absorbance.
Clause 11. The apparatus of any one of clauses 1-10, wherein the plurality of lower conduits is in fluid communication with a separation means.
Clause 12. A cell culture system comprising a plurality of the apparata of any one of clauses 1-11 fluidly connected to each other.
Clause 13. A method or means for cultivating cells and producing cellular products, the method comprising introducing a plurality of cells into the bioreactor of any one of clauses 1-11 or the cell culture system of clause 12 and incubating the cells under conditions sufficient for growth.
Clause 14. The method or means of clause 13, wherein cellular products produced by the cells are isolated.
Clause 15. A biological product produced by the method or means of clause 13 or 14.

### EXAMPLES

### Example 1

The following is an exemplary protocol for use of the modular cell culture bioreactor apparatus described herein for large-scale production of insulin in CHO cells. CHO cells and the cellular product insulin are described in this example however, the apparatus is effective in large-scale production of other cellular products from any cell line.

CHO cells will be transfected with a mammalian expression vector carrying cDNA for the human insulin gene under the control of a promoter. The vector may also comprise a marker for selection of the CHO cells that express the gene such as a fluorescent tag. Stable transfectants will be isolated and characterized in regard to insulin transcription, translation, processing, and secretion. The CHO cells that effectively express and secrete insulin will be mixed with a bioink or seeded on the bioink and bioprinted in the cylindrical wells (cell chambers) (FIG. 1C). Bioinks are natural or synthetic biomaterials that mimic the extracellular matrix (ECM) to support the adhesion, proliferation, or differentiation of the cells, and are customized for the specific cell type. The bioink that will be used in this example is a low-density collagen-embedding gel.

A permeable membrane will be placed on the upper and lower sides of the three-dimensional cell distribution (FIG. 1C). Optionally, a layer of epithelial cells will be disposed upon the three-dimensional distribution of cells and under the upper permeable membrane to feed the CHO cells. The three-dimensional cell structure (e.g., cell chamber) comprising the insulin-producing CHO cells will be placed into the bioreactor (FIG. 1A). The bioreactor comprising the cells will be placed into a cell culture apparatus (FIG. 2). The cell culture apparatus includes an upper flow chamber (feeding) and a lower flow chamber (collection), a plurality of upper conduits fluidly connecting the upper flow chamber with several upper reservoirs that separately contain nutrient media and pH adjusting agents, and a plurality of lower conduits fluidly connects the lower flow chamber with a lower reservoir containing a buffer (FIG. 3-4). The cell culture apparatus will be fluidly connected with other cell culture apparatuses to create a cell culture system. The upper flow chamber will maintain the cells while the lower flow chamber will collect the cell products.

The cells in the culture system will be incubated under conditions sufficient for growth. Alternatively, fast-maturing cells can be inserted in the bioreactor ready for product collection. Throughout incubation, the secreted products and accompanying metabolites from the cells will be collected through the lower fluid, which will be carried out to a tangential flow separation column for product separation and further sample processing (FIG. 3-4). The nutrient media will push the cell products from the upper part of the apparatus to the lower part of the apparatus where the outlet is as a result of a permanent gradient between the nutrient media in the upper conduits and the buffer in the lower conduits. Then, the cell products (e.g., insulin) will be isolated and purified from the supernatant collected from the outlet, first by separating products from the collecting media using tangential flow and then by other methods such as immunoprecipitation, chromatography, or other methods known in the art.

What is claimed:

1. A modular cell culture bioreactor apparatus comprising:
a plurality of cell chambers disposed between an upper flow chamber and a lower flow chamber;
a plurality of lower conduits fluidly connecting the lower flow chamber with one or more lower reservoirs and a plurality of upper conduits fluidly connecting the upper flow chamber with one or more upper reservoirs;
one or more pumps fluidly connected through the plurality of conduits with the one or more reservoirs and with the upper and lower flow chambers;
wherein each individual cell chamber comprises a lower permeable membrane in fluid communication with the lower flow chamber; a three-dimensional distribution of cells; and an upper permeable membrane in fluid communication with the upper flow chamber.

2. The apparatus of claim 1, wherein the upper flow chamber, upper conduits, and at least one upper reservoir comprises a culture medium.

3. The apparatus of claim 1, wherein the one or more upper reservoirs comprise a culture medium, a cell feed, a cell supplement, a buffering agent, oxygen or carbon dioxide gases, or antibiotics.

4. The apparatus of claim 1, wherein the lower flow chamber, lower conduits, and at least one lower reservoir comprises a buffered solution.

5. The apparatus of claim 1, wherein the three-dimensional distribution of cells is embedded in a low-density gel.

6. The apparatus of claim 1, wherein the three-dimensional distribution of cells is deposited with a 3D bioprinter.

7. The apparatus of claim 1, wherein the three-dimensional distribution of cells is in solution or suspension.

8. The apparatus of claim 1, wherein the cells comprise human, mammalian, insect, archaea, bacteria, cancers, genetically modified cells, or transformed or transfected cells.

9. The apparatus of claim 1, wherein the cell chamber further comprises a layer of epithelial cells disposed upon the three-dimensional distribution of cells and under the upper permeable membrane.

10. The apparatus of claim 1, wherein the plurality of lower conduits and/or a plurality of upper conduits comprise fluid or gas outlets, fluid or gas inlets, fluid waste ports, and sensors for temperature, pH, dissolved oxygen, or UV/Vis absorbance.

11. The apparatus of claim 1, wherein the plurality of lower conduits is in fluid communication with a separation means.

12. A cell culture system comprising a plurality of the apparata of claim 1 fluidly connected to each other.

13. A method or means for cultivating cells and producing cellular products, the method comprising introducing a plurality of cells into the bioreactor of claim 1 and incubating the cells under conditions sufficient for growth.

14. The method or means of claim 13, wherein cellular products produced by the cells are isolated.

15. A biological product produced by the method or means of claim 13.
